(19) United States
(12) Patent Application Publication
(10) Pub. No.: US 2011/0082550 A1
(43) Pub. Date: Apr. 7, 2011

(54) INTERVERTEBRAL FIXATION DEVICE

(76) Inventor: An-Shih YEH, Taipei City (TW)

(21) Appl. No.: 12/574,901

(22) Filed: Oct. 7, 2009

(51) Int. Cl. A61F 2/44; A61B 17/70

(52) U.S. Cl. 623/17.11; 606/246

(57) ABSTRACT

This invention discloses an intervertebral fixation device including a plate member, a body, and at least one bending portion disposed on the plate member. The thickness of the bending portion is slightly smaller than the thickness of the plate member and ear portions, such that the bending portion is provided for flexibly adjusting a curvature of the plate member of the intervertebral fixation device, and an ear portion and another ear portion on the plate member can be adjusted flexibly to be attached to the two cervical vertebrae, and the intervertebral fixation device can match with the physiological curvature of each patient's cervical vertebrae, so as to overcome the shortcomings of a conventional intervertebral fixation device having a protrusion after a surgical operation is performed, and prevent a locking element secured to a conventional intervertebral fixation device from being loosened, and avoid another surgical operation of a patient.

### BACKGROUND OF THE INVENTION

1. Field of Invention

The present invention relates to an intervertebral fixation device, and more particularly to an intervertebral fixation device having at least one bending portion disposed on a plate member of the intervertebral fixation device, and the thickness of the bending portion is slightly smaller than the thickness of the plate member and ear portions, and the bending portion is provided for flexibly adjusting the curvature of the intervertebral fixation device to fit a physiological curvature of a patient's cervical spine, so as to overcome the shortcomings of conventional intervertebral fixation devices that may cause a bump after a surgery operation takes place, a locking element of the conventional intervertebral fixation devices being loosened or fallen out, and may require a patient to take another surgical operation again.

2. Description of Related Art

Human body is supported by skeleton inside the body, so that muscles and blood vessels will not be all piled up together due to gravitational force, and a spinal cord extended downwardly from brain to buttock is the most important part of human skeleton. The spinal cord contains spinal nerves and is composed of vertebrae, such that if any of our vertebrae is shifted, then we will be unable to move conveniently or even unable to move at all in a serious case.

In our spinal cord, cervical vertebrae at our neck are small and fragile, and the structure of the cervical vertebrae is similar to the structure of the spinal cord, except the cervical vertebrae are smaller in size and more fragile.

However, a conventional intervertebral fixation device is generally used for correcting a vertebra displacement, but the curvature and the size of each patient's cervical vertebrae are different, so that after a patient uses the conventional intervertebral fixation device for correction or fixation, the conventional intervertebral fixation device may be protruded to form a bump or a locking element locked at the conventional intervertebral fixation device may be loosened or fallen off after a surgical operation is performed.

With reference to FIG. 1 for a perspective view of a conventional intervertebral fixation device 1, the intervertebral fixation device 1 is composed of a plate member 11 and a body 12 integrated with one another as a whole, wherein a lug 111 is extended horizontally outward from an end of the plate member 11, and the lug 111 has a hole 1110, and another lug 112 is extended horizontally outward from another end of the plate member 11, and the lug 112 has a hole 1120, and a body 12 is extended from a side of the plate member 11, such that the body 12 and the plate member 11 can be integrated as a whole, and a plurality of latching teeth 121, 122 are disposed on both top and bottom sides of the body 12 respectively and engaged with a contact surface (not shown in the figure) between two cervical vertebrae, and an opening 123 is formed on each surface of the body 12 without having the plate member 11, and the openings 123 are provided for placing soft tissues including implant bones.

After the intervertebral fixation device 1 is installed between two cervical vertebrae, the two locking elements 21, 22 can be passed through a hole 1110 and another hole 1120 of the lugs 111, 112 respectively, and then secured to a central position (not shown in the figure) of the two cervical vertebrae. Since the cervical vertebrae of a human body come with a specific curvature, and the lugs 111, 112 on the plate member 11 of the intervertebral fixation device 1 are installed horizontally, therefore the intervertebral fixation device 1 cannot be matched with the physiological curvature of the cervical vertebrae after the intervertebral fixation device 1 is installed and secured between the two cervical vertebrae. As a result, the intervertebral fixation device 1 will be protruded to form a bump after the surgical operation takes place, or the locking elements 21, 22 of the intervertebral fixation device 1 will be loosened or fallen off, such that the patient has to take another surgical operation again.

With reference to FIG. 2 for a perspective view of another conventional intervertebral fixation device 3, the other intervertebral fixation device 3 is composed of a plate member 31 and a body 32 integrated with one another, wherein a lug 311 is extended laterally and slantingly outward from an end of the plate member 31, and the lug 311 has a hole 3110, and another lug 312 is extended laterally and slantingly outward from another end of the plate member 31, and the other lug has a hole 3120, and an opening 313 is formed at a central position of the plate member 31, and a body 32 is extended from a side of the plate member 31, such that the body 32 and the plate member 31 are integrated with one another as a whole, and the body 32 is substantially in a U-shaped hollow form, and a top side 321 of the body 32 is substantially in an arc shape to fit the physiological curvature of a contact surface (not shown in the figure) between two cervical vertebrae.

After the other intervertebral fixation device 3 is installed between two cervical vertebrae, two locking elements 41, 42 are passed through a hole 3110 and another hole 3120 of the lugs 311, 312 respectively and secured to a central positions (not shown in the figure) of the two cervical vertebrae, wherein a lug 311 and another lug 312 on the plate member 31 are designed with a curvature tilted towards the body 32 to fit the curvature of the two connected cervical vertebrae. However, the other intervertebral fixation device 3 is made of a metal, so that the elasticity is not as good, and the aslant curvature of the lugs 311, 312 may not be able to match with the physiological curvature of each patient's cervical vertebrae. When the other intervertebral fixation device 3 is installed, the other intervertebral fixation device 3 may be unable to match with the physiological curvature of the patient's cervical vertebrae. Similarly, the other intervertebral fixation device 3 also forms a bump after the surgical operation takes place, or the locking elements 41, 42 secured to the other intervertebral fixation device 3 will be loosened or fallen off, such that the patient has to take another surgical operation again.

In view of the aforementioned shortcomings of the conventional intervertebral fixation devices 1, 3 that cannot match the physiological curvature of each patient's cervical vertebrae, and thus the present invention provides a better intervertebral fixation device to meet user requirements.

### SUMMARY OF THE INVENTION

Therefore, it is a primary objective of the present invention to provide an intervertebral fixation device, comprising at least one bending portion disposed on a plate member and provided for flexibly adjusting the curvature of the intervertebral fixation device, such that the intervertebral fixation device is matched with the physiological curvature of each patient's cervical vertebrae to overcome the shortcomings of the conventional intervertebral fixation device having a bump after a surgical operation takes place, and prevent a locking element secured to the conventional intervertebral fixation device from being loosened or fallen off, and avoid taking another surgical operation again.

### BRIEF DESCRIPTION OF THE DRAWINGS

The invention, as well as its many advantages, may be further understood by the following detailed description and drawings in which:

FIG. 1 is a perspective view of a conventional intervertebral fixation device;

FIG. 2 is a perspective view of another conventional intervertebral fixation device;

FIG. 3 is a perspective view of a plate member of an intervertebral fixation device in accordance with the present invention;

FIG. 4 is a perspective view of another side of a plate member of an intervertebral fixation device in accordance with the present invention;

FIG. 5 is a perspective view of a body of an intervertebral fixation device in accordance with the present invention; and

FIG. 6 is a perspective view of assembling an intervertebral fixation device in accordance with the present invention.

### DETAILED DESCRIPTION OF THE INVENTION

To make it easier for our examiner to understand the technical measures and the operating procedure of the present invention, we use preferred embodiments together with the attached drawings for the detailed description of the present invention.

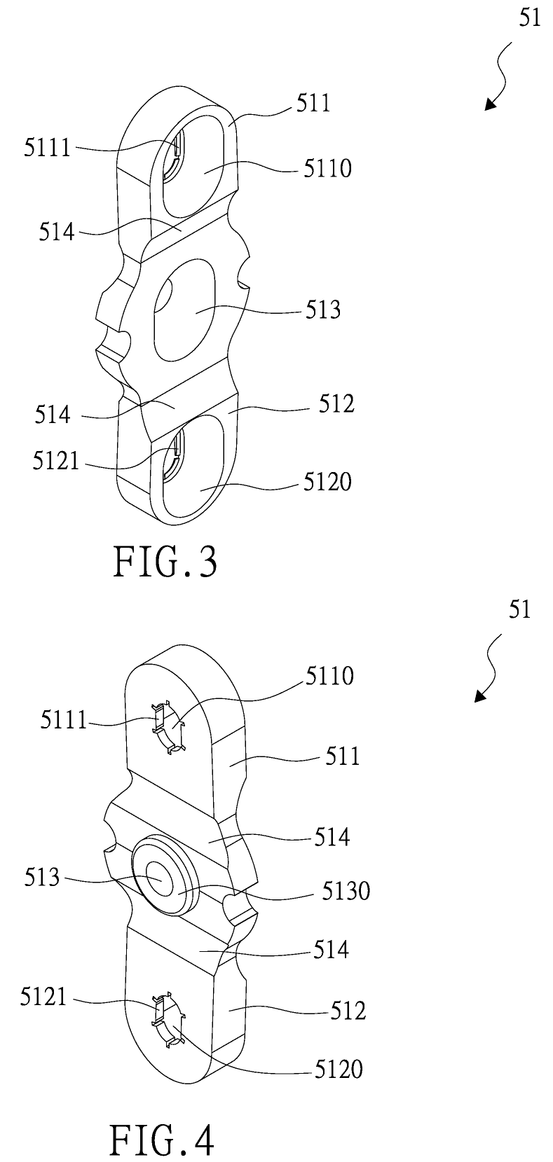

With reference to FIGS. 3 to 6 for perspective views of a plate member of an intervertebral fixation device 5 and a perspective view of assembling the intervertebral fixation device 5 in accordance with the present invention respectively, the intervertebral fixation device 5 comprises a plate member 51 and a body 52.

An ear portion 511 is extended upwardly from an end of the plate member 51, and a first hole 5110 is formed at a central position of the ear portion 511, and another ear portion 512 is extended downwardly from another end of the body 51, and a second hole 5120 is formed at a central position of the other ear portion 512, and a plurality of wing plates 5111, 5121 are disposed on a side of the first hole 5110 and the second hole 5120 respectively, and a third hole 513 is formed at a central position of the plate member 51, and a convex ring 5130 is disposed at the third hole 513 and corresponding to a side of the body 52 (wherein the convex ring 5130 of this embodiment is integrally formed with the plate member 51, and can be a washer), and at least one bending portion 514 (as shown in FIGS. 3 and 4) is disposed on the plate member 51 and has a thickness slightly smaller than the thickness of the plate member 51 and the ear portions 511, 512 for flexibly bending the intervertebral fixation device 5, and the plate member 51 is made of an elastic plastic material.

In the present invention, the bending portion 514 can be disposed on a side, another side or both sides of the plate member 51 for flexibly bending the intervertebral fixation device, such that the intervertebral fixation device 5 can be matched with the physiological curvature of each patient's cervical vertebrae.

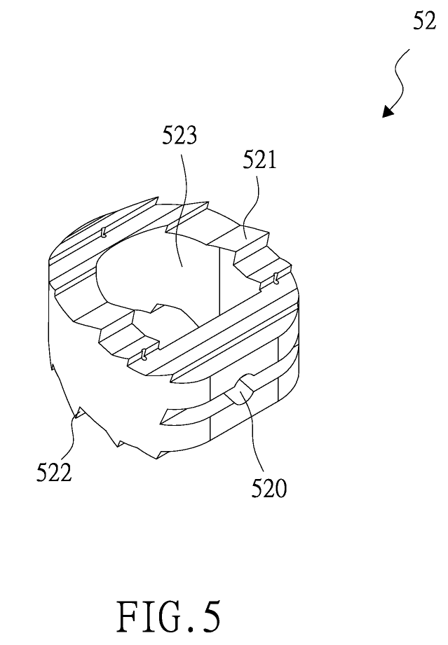

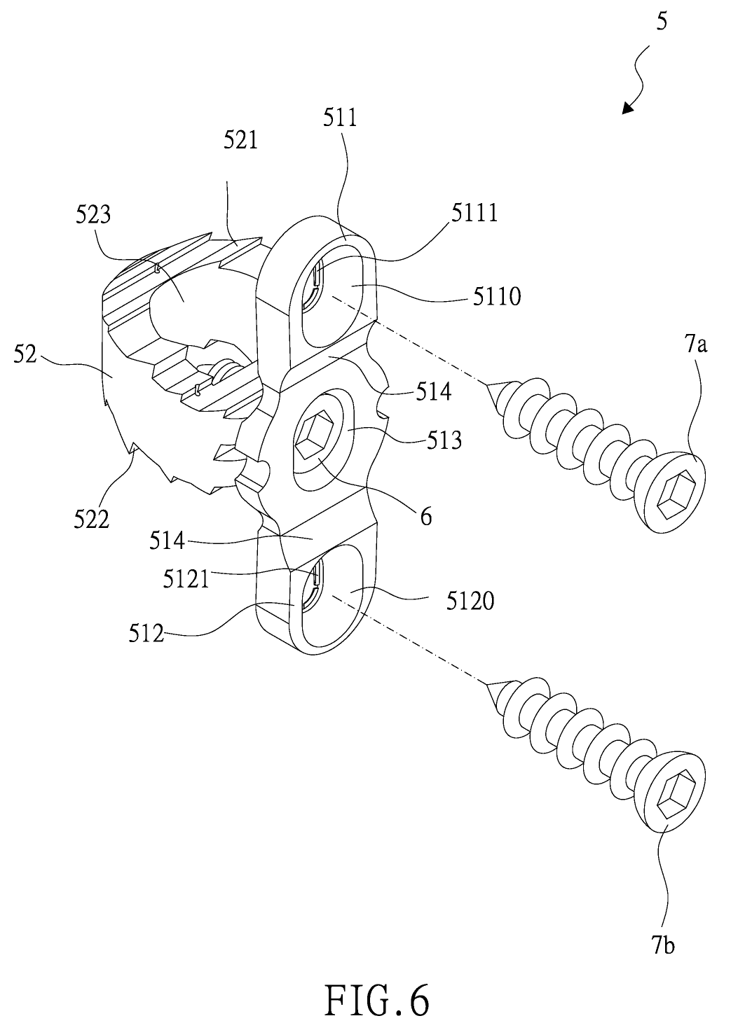

A screw hole 520 is formed on a side of the body 52 (as shown in FIG. 5) and secured with a third hole 513 of the plate member 51 by a locking element 6 (as shown in FIG. 6), and the crew hole 520 is in contact with the convex ring 5130 for combining the body 52 with the plate member 51, such that the body 52 is situated on a side of the plate member 51 and between the ear portions 511, 512. The way of combining the plate member 51 and the body 52 is not limited to the aforementioned arrangement only, but the plate member 51 and the body 52 can be integrally formed.

A plurality of latching teeth 521, 522 are disposed at top and bottom sides of the body 52 respectively, and a penetrating hole 523 is penetrated from a central position of the top side to a central position of the bottom side (as shown in FIG. 5), and provided for placing an implant bone tissue of a patient.

With the aforementioned elements, the body 52 is installed between two cervical vertebrae for the use of the intervertebral fixation device 5, other locking elements 7a, 7b (as shown in FIG. 6) are passed through holes 5110, 5120 of the ear portions 511, 512 respectively, and secured to a central position (not shown in the figure) of the two cervical vertebrae. A plurality of wing plates 5111 of the holes 5110, 5120 can be used for preventing the other locking elements 7a, 7b from being loosened or fallen off. The bending portion 514 is provided for flexibly adjusting the curvature of the plate member 51 of the intervertebral fixation device 5 to match the physiological curvature of a patient's cervical vertebrae, so as to effectively overcome the shortcomings of the conventional intervertebral fixation device 5 having a protrusion after the surgical operation takes place, and prevent other locking elements 7a, 7b secured to the conventional intervertebral fixation device 5 from being loosened or fallen off, and avoid another surgical operation of a patient for the treatment of a pathological change of cervical vertebrae.

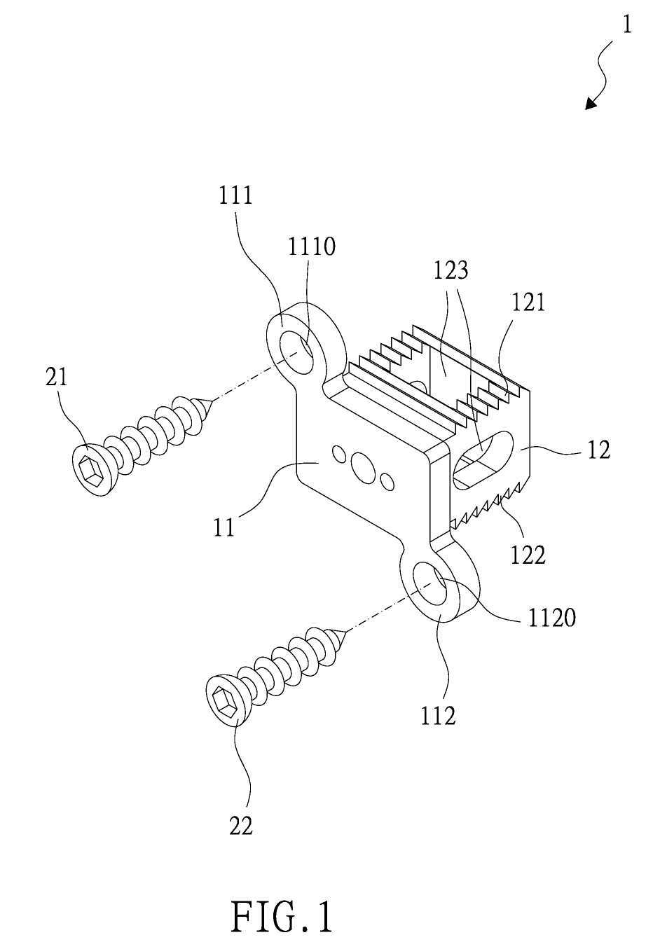

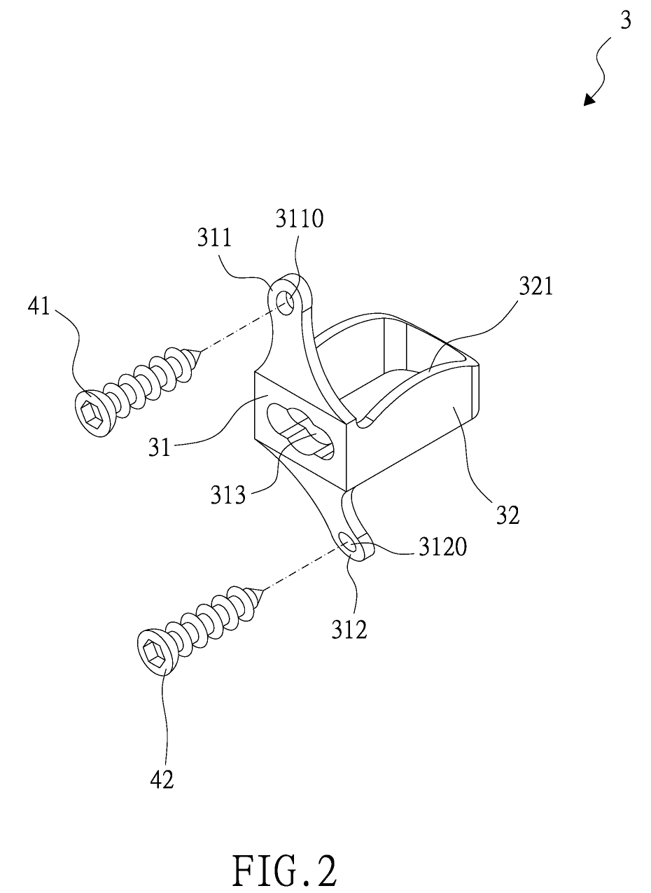

Unlike the prior art, the technical characteristics of the present invention reside on that the intervertebral fixation device 5 installed on the plate member 51 comprises at least one bending portion 514 having a thickness slightly smaller than the thickness of the plate member 51 and the ear portions 511, 512. The bending portion 514 is provided for giving a better elasticity of the intervertebral fixation device 5 and allowing users to flexibly adjust the curvature of the ear portions 511, 512 of the intervertebral fixation device 5 to match with the physiological curvature of each patient's cervical vertebrae. The invention can overcome the shortcomings of the conventional intervertebral fixation device 1, 3 whose lugs 111, 112, 311, 312 (as shown in FIGS. 1 and 2) are not installed horizontally and cannot match with the physiological curvature of the cervical vertebrae. In other words, the slant curvature may not be able to match with the overall curvature of each patient's cervical vertebrae, and thus causes many problems after the surgical operation takes place. Obviously, the invention improves over the prior art and complies with the patent application requirements.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. An intervertebral fixation device, comprising:
a plate member, having an ear portion disposed at an end of said plate member, a first hole formed at a central position of said ear portion, another ear portion disposed on another end of said plate member, a second hole formed at a central position of said other ear portion, a plurality of wing plates installed on a lateral side of said first hole and said second hole respectively, a third hole formed at a central position of said plate member, and at least one bending portion disposed on the plate member, and the thickness of said bending portion being slightly smaller than the thickness of said plate member and said ear portions; and
a body, situated on a surface of said plate member and between said ear portions, having a screw hole disposed at a position corresponding to said third hole of said plate member, and said screw hole being provided for locking a locking element therein.

2. The intervertebral fixation device of claim 1, wherein said plate member is made of an elastic plastic material.

3. The intervertebral fixation device of claim 2, wherein said wing plates are provided for preventing another locking element from being loosened or fallen out after said locking element is secured.

4. The intervertebral fixation device of claim 3, wherein said third hole includes a convex ring disposed at a position corresponding to a side of said body and in contact with said screw hole.

5. The intervertebral fixation device of claim 4, wherein said convex ring is integrated with the plate member as a whole and selected from one of the washers.

6. The intervertebral fixation device of claim 5, wherein said at least one bending portion is disposed on a side, another side or both sides of said plate member.

7. The intervertebral fixation device of claim 6, wherein said intervertebral fixation device provides a curvature through said at least one bending portion for adjusting an elasticity of said intervertebral fixation device.

8. The intervertebral fixation device of claim 7, wherein said plate member and said body are integrated with each other as a whole.
